(19) United States
(12) Patent Application Publication
(10) Pub. No.: US 2026/0221226 A1
(43) Pub. Date: Jul. 30, 2026

(54) PREDICTIVE METHOD FOR DETERMINING THE DIGENIC MECHANISM OF A DIGENIC COMBINATION RECOGNIZED AS PATHOGENIC

(71) Applicant: ENGENOME S.R.L., Pavia (IT)

(72) Inventors: Giovanna NICORA, Pavia (IT); Federica DE PAOLI, Pavia (IT); Susanna ZUCCA, Pavia (IT); Ivan LIMONGELLI, Pavia (IT); Riccardo BELLAZZI, Pavia (IT); Paolo MAGNI, Pavia (IT); Ettore RIZZO, Pavia (IT)

(21) Appl. No.: 19/147,641

(22) PCT Filed: Jan. 9, 2024
(86) PCT No.: PCT/IB2024/050205

§ 371 Date: Jul. 11, 2025

(30) Foreign Application Priority Data
Jan. 13, 2023 (IT) 102023000000363

(51) Int. Cl. G16B 20/20; G06N 20/00; G16B 40/20

(52) U.S. Cl. CPC G16B 20/20; G06N 20/00; G16B 40/20

(57) ABSTRACT

A computer-implemented method predicts/determines the digenic mechanism of a digenic combination recognized as pathogenic, in observed phenotypes. The method applied to a digenic combination recognized as pathogenic based on a pathogenicity determination performed by a trained algorithm. The pathogenicity determination is based on at least the following: variant-level features representative of pathogenicity of the single variants forming the digenic combination; gene-level features representing interaction between the genes forming the digenic combination and/or a priori property features of each gene of the digenic pair; phenotype-level features representing gene-phenotype association, to measure superimposability of patient phenotypic traits to already associated phenotypes. The digenic mechanism is “Dual Diagnosis” if each mutated gene individually causes a respective disease. First, second and third weight parameters are determined by Explainable Artificial Intelligence/XAI. Machine learning techniques/models determine the digenic mechanism, and then recognizes whether the digenic mechanism is “Dual Diagnosis” based on the weight parameters.

### CROSS-REFERENCE TO RELATED APPLICATIONS

This application is a National Stage Application of PCT/IB2024/050205, filed Jan. 9, 2024, which claims benefit of priority to IT patent application No. 102023000000363 filed on Jan. 13, 2023, and which applications are incorporated herein by reference. To the extent appropriate, a claim of priority is made to each of the above disclosed applications.

### FIELD OF APPLICATION

The present invention relates to a predictive method for determining the digenic mechanism of a digenic combination recognized as pathogenic.

Therefore, the general technical field of the present invention is that of predictive methods, performed by electronic computation, used in the context of genomics and/or medical genetic research to support predictive prognoses.

### PRIOR ART

The hypothesis of digenic inheritance, in which the development of a disease occurs in the presence of two mutated genes, is increasingly being considered by geneticists to solve undiagnosed cases.

The broadest definition of “digenic inheritance” corresponds to three different categories of digenicity, caused by three different digenic mechanisms (i.e., interaction between the two different genes).
1. True digenic, in which both genes must be mutated simultaneously for the disease to occur. If a patient has only one of the two mutated genes, he/she will not develop the phenotypes associated with the disease.
2. “Composite” or “Modifier”: in this case the mutation of one of the two genes (“major gene”) causes the occurrence of the disease in the patient, while the second mutated gene, which individually has no phenotypic effects, if co-present with the “major gene” has a “modulating” effect on the emergence of the disease. Typically, the modulator determines a greater severity of the disease, or the onset of symptoms at a younger age.
3. Dual Diagnosis (a mechanism also commonly known as “Dual Molecular Diagnosis”): in this latter case, a patient suffers from two different genetic diseases simultaneously, each caused by a single mutated gene. The patient will tend to show phenotypes related to both diseases, often making diagnosis difficult.

According to this sub-categorization, variants which are part of a pathogenic digenic combination can also have very different features.

In the aforesaid case of “True Digenic”, the two mutated genes, taken individually, are not causative of phenotype. The underlying molecular mechanism includes a close interaction between the two genes involved, often belonging to the same biological pathway.

The term “gene interaction” refers to two genes which, for example, are part of the same biological pathway, i.e., they cooperate within the cell to the production of some fundamental molecules for the cell itself, such as proteins.

As a result, variants on two interacting genes can compromise the biological mechanism in which both genes are involved, leading to the development of a genetic disease.

On the other hand, in the other aforesaid case of the “Composite” mechanism, one of the two genes considered individually is affected by pathogenic variants causing the disease, while the second is not. Also in this configuration, it is very likely that the two genes interact with each other or have effects related to common biological mechanisms.

In the other case of “Dual Diagnosis”, the two mutated genes cause two diseases independently. Therefore, it is expected that the two variants taken individually are highly pathogenic, while the genes have no particular interaction with each other.

Once it has been ascertained (for example, by means of any determination or prediction method) that a particular combination of variants represents a pathogenic digenic combination, it can be useful to understand which digenic mechanism is occurring in the case in hand (True Digenic, Composite or Dual Diagnosis), so as to provide the geneticist with additional information to define the treatment strategy, such as the most effective therapeutic choice for that patient.

While known predictive methods exist for predicting the pathogenicity of a digenic pair (for example, the proprietary solution developed by the Applicant, and described in international patent application WO 2022/195507 A1, to the same Applicant, is mentioned here), the need for methods to determine, in a predictive manner, the digenic mechanism through which a digenic pair (or, more generally, a digenic combination) operates still remains largely unmet.

In this respect, the generic approach of applying Machine Learning or Artificial Intelligence techniques for this purpose (as well as for many other diagnostic predictive purposes) can be considered known, without however, as far as the Applicant is aware, solutions being available which fully meet the need to effectively and reliably predict the digenic mechanism through which a digenic combination considered pathogenic operates, and therefore to draw therefrom the important diagnostic evaluations mentioned above.

### SUMMARY OF THE INVENTION

It is the object of the present invention to provide a computer-implemented predictive method for determining the digenic mechanism of a digenic combination recognized as pathogenic, which allows at least partially overcoming the drawbacks described above with reference to the prior art and responding to the aforesaid needs particularly felt in the technical field considered.

It is another object of the present invention to provide a corresponding method for determining the pathogenicity of combinations of digenic variants and for classifying digenic variants recognized as pathogenic.

### BRIEF DESCRIPTION OF THE DRAWINGS

Further features and advantages of the method according to the invention will become apparent from the following description of preferred embodiments, given by way of non-limiting indication, with reference to the accompanying drawings, in which:

FIG. 1 depicts a simplified block diagram showing an embodiment of the method according to the invention.

### DETAILED DESCRIPTION

A computer-implemented predictive method for predicting and/or determining the digenic mechanism of a digenic combination recognized as pathogenic, in relation to a disease, or in relation to a set of phenotypes observed in an individual, is described.

Note that the aforesaid digenic combination refers to variants related to two genes, referring to mutations present in one or both alleles of a respective gene of at least two genes, each of the genes being associated with two respective alleles. The method applies, in particular, to a digenic combination which has been recognized as pathogenic based on a pathogenicity determination carried out by a trained algorithm trained by means of artificial intelligence and/or machine learning techniques based on a training dataset of known cases, wherein the pathogenicity determination has been performed based on at least the following features:
variant-level features, representative of the pathogenicity of the single variants forming the digenic combination;
gene-level features, representative of the interaction between the genes forming the digenic combination and/or a priori property features of each of the two genes of the digenic pair;
phenotype-level features, representative of gene-phenotype association, calculated individually for each of the genes considered, and adapted to measure how much the phenotypic traits of the patient are superimposable to phenotypes already known to be associated with the single gene.

The recognition of the digenic mechanism comprises recognizing whether the digenic mechanism is “Dual Diagnosis” or not, in which the digenic mechanism (according to a term commonly used in the technical field considered) is defined as “Dual Diagnosis” if each mutated gene individually causes a respective disease.

The method first comprises a step of determining, by means of Explainable Artificial Intelligence, XAI, techniques, a first weight parameter vlc, a second weight parameter, glc, and a third weight parameter, phlc.

The first vlc weight parameter (which will also be referred to as “variant-level contribution” in some embodiments illustrated below) is representative of the contribution of variant-level features on the pathogenicity determination of the digenic combination.

The second weight parameter glc (which will also be referred to as “gene-level contribution” in some embodiments illustrated below) is representative of the contribution of gene-level features on the pathogenicity determination of the digenic combination.

The third weight parameter phlc (which will also be referred to as “phenotype-level contribution” in some embodiments illustrated below) is representative of the contribution of phenotype-level features on the pathogenicity determination of the digenic combination.

The method then provides determining, by means of machine learning techniques or models, the digenic mechanism of the digenic combination, and then recognizing whether the digenic mechanism is “Dual Diagnosis” or not, based on the aforesaid first weight parameter vlc, second weight parameter glc and third weight parameter phlc, determined by Explainable Artificial Intelligence, XAI, techniques.

Note that, according to possible implementations, the method is applied to a “digenic combination” comprising a number of variants which can typically be two variants (in such a case, the digenic combination is actually a “digenic pair”) or three variants or four variants, if each allele has a variant.

Further note that the definition known per se as “Explainable Artificial Intelligence” (XAI) is intended as a set of approaches aiming at making the classification process performed by an Artificial Intelligence or Machine Learning algorithm understandable.

The property of “explainability” is an important requirement to promote confidence in the use of Artificial Intelligence. Moreover, it is indicated as a fundamental property by various regulatory bodies for the application of Artificial Intelligence in areas where decisions adopted based on predictions made by Artificial Intelligence can significantly impact people's lives (as in the case of medicine).

There are known “Explainable Artificial Intelligence” approaches called “local”, the ambition of which is to explain why a Machine Learning algorithm has predicted a certain classification for a given example, highlighting for example the “features” (in accordance with a common term in the technical field considered here) of that given example which led the algorithm to assign a certain class.

In the following of this description, there will be illustrated further examples and details of the use of “Explainable Artificial Intelligence” (XAI) for the purpose of predicting the digenic mechanism with which a digenic combination operates, or to classify a digenic combination, previously recognized as pathological, in one of the aforesaid sub-categories “True Digenic” or “Composite” and/or “Dual Diagnosis”.

In accordance with an embodiment, the aforesaid step of determining, by means of Explainable Artificial Intelligence, XAI, techniques, a first weight parameter vlc, a second weight parameter glc and a third weight parameter phlc comprises:
calculating the first weight parameter vlc as the Shapley value of the variant-level features, indicative of the contribution of the variant-level features to the predicted pathogenicity probability of the digenic combination;
calculating the second weight parameter glc as the Shapley value of the gene-level features, indicative of the contribution of the gene-level features to the predicted pathogenicity probability of the digenic combination;
calculating the third weight parameter phlc as the Shapley value of the phenotype-level features, indicative of the contribution of the phenotype-level features to the predicted pathogenicity probability of the digenic combination.

With reference to the aforementioned “Shapley values”, which are also known per se, the following remarks are provided.

In game theory, which deals with modeling different situations of strategic interaction, Shapley's values represent a “reward” which is given to each player belonging to a coalition, based on the contribution of the player himself to the achievement of the objectives. Therefore, the “reward” is distributed to the players of the same coalition in proportion to their contribution (see for example Robert J. Aumann and Lloyd S. Shapley “Values of non-atomic games”, Princeton University Press, Princeton, 1974).

In the context of Artificial Intelligence, Shapley's values are used to understand the contribution of each feature (player) to the prediction (which represents the objective achieved) of a single example (local explainability-see for example S.Lundberg, S. I.Lee, “A Unified Approach to Interpreting Model Predictions”, arXiv: 1705.07874 [cs, stat], November 2017, http://arxiv.org/abs/1705.07874).

In short, given a certain probability of classification predicted for a given example, characterized by a set of features, the “Shapley value” of each feature is calculated as the marginal contribution of that feature in the prediction of probability. This contribution is calculated by verifying the aforementioned probability variation using “fictitious” examples in which the value of the feature in hand is kept fixed, while the values of the other features change.

According to Shapley's theory, the predicted probability is “broken down” into N values (where N is equal to the number of features) each of which represents the contribution of each feature to the classification. For a given example classified by a Machine Learning type algorithm, the higher the Shapley value for a certain feature, the more the value of that feature was important in determining the classification of that specific example.

In accordance with an embodiment, the method comprises the following further steps:
calculating, in a preliminary training step on a training dataset, a first conditional probability of “Dual Diagnosis” digenic combination p(DM|variant_level_contribution) based on the frequency of digenic training combinations indicated in the training dataset as of “Dual Diagnosis” type given a first parameter value variant_level_contribution corresponding to the first weight parameter vlc (calculated or determined, as previously described, by means of XAI techniques, for each digenic training combination belonging to the training dataset), or a discretization of such a first weight parameter into a limited number of discrete levels;
calculating, in a preliminary training step on the training dataset, a second conditional probability of “Dual Diagnosis” digenic combination p(DM|gene_level_contribution) based on the frequency of digenic training combinations indicated in the training dataset as of “Dual Diagnosis” type given a second parameter value gene level_contribution corresponding to the second weight parameter glc (calculated or determined, as previously described, by means of XAI techniques, for each digenic training combination belonging to the training dataset), or a discretization of such a second weight parameter into a limited number of discrete levels;
calculating, in a preliminary training step on the training dataset, a third conditional probability of “Dual Diagnosis” digenic combination p(DM|phenotype_level_contribution) based on the frequency of digenic training combinations indicated in the training dataset as of “Dual Diagnosis” type given a third parameter value phenotype level_contribution corresponding to the third weight parameter phlc (calculated or determined, as previously described, by means of XAI techniques, for each digenic training combination belonging to the training dataset), or a discretization of such a third parameter into a limited number of discrete levels.

In such an embodiment, the step of determining the digenic mechanism of a digenic combination to be classified, in an operating step of the method, comprises determining the digenic mechanism of the digenic combination based on the aforesaid first conditional probability, second conditional probability, and third conditional probability.

According to an implementation option of the aforesaid embodiment, the step of determining the digenic mechanism of the digenic combination comprises calculating a score proportional to the probability that the digenic mechanism is “Dual Diagnosis” by means of a Naive Bayes-type machine-learning model, based on the training dataset, and then determining that the digenic mechanism is “Dual Diagnosis” if the aforesaid score proportional to the probability P (DM|Xi) that the digenic mechanism is “Dual Diagnosis” is above a certain threshold.

In particular, the calculation of the score proportional to the probability that the digenic mechanism is “Dual Diagnosis” by means of a Naive Bayes-type machine-learning model is carried out based on the following formula [1]:

$$p ( DM \vert {X}_{i} ) ∼ p ( DM ) * \frac{p ( DM \vert gene\_level {\_contribution}_{i} )}{p ( DM )} * \frac{p ( DM \vert variant\_level {\_contribution}_{i} )}{p ( DM )} * \frac{p ( DM \vert pheno\_level {\_contribution}_{i} )}{p ( DM )}$$

in which:
p(DM) is the total probability that the digenic mechanism is “Dual Diagnosis” calculated on the training dataset as well;
variant_level_contribution corresponds to the first weight parameter vlc or a discretization of the first weight parameter into a limited number of discrete levels;
gene_level_contribution corresponds to the second weight parameter (glc) or a discretization of the second weight parameter into a limited number of discrete levels;
phenotype_level_contribution corresponds to the third weight parameter (phlc) or a discretization of the third weight parameter into a limited number of discrete levels.

According to a particular implementation option of the method, the predicted score is normalized between 0 and 1, to obtain a normalized score (P_{FINAL}), by means of the following formula:

$${P}_{FINAL} ( DM \vert {X}_{i} ) = \frac{P ( DM \vert {X}_{i} )}{P ( DM \vert {X}_{i} ) + P ( NoDM \vert {X}_{i} )}$$

In such a case, the aforesaid step of determining that the digenic mechanism is “Dual Diagnosis” comprises comparing such a normalized score (P_{FINAL}) with a normalized threshold.

According to an implementation example, the aforesaid normalized threshold is 0.5.

In accordance with an implementation option, each of the aforesaid variant_level_contribution, gene_level_contribution, phenotype_level_contribution parameters corresponds to a respective discretization of the first weight parameter, the second weight parameter, the third weight parameter, respectively.

According to an even more detailed implementation option, each of the aforesaid variant_level_contribution, gene_level_contribution, phenotype_level_contribution parameters corresponds to a respective discretization of the first weight parameter, the second weight parameter, the third weight parameter into four discrete levels: low, medium-low, medium-high, high.

Further embodiments of the method, employing the “Naive Bayes” approach, will be described below in this description, with more exemplary details.

As for the “Naive Bayes” approach, used in the embodiment of the method shown above, it can be observed that one of the positive aspects of the “Naive Bayes” is of being “interpretable a priori”, meaning that it allows understanding which are the most important features for the estimation of the classification, based on the formula explained above. Naive Bayes falls within the Machine Learning algorithms when the conditional probabilities of a class given a value of a feature are calculated from a training dataset.

In accordance with another embodiment, the method further comprises the steps of:
calculating a first contribution β₁*variant_level_contribution, where β₁ is a parameter estimated in a preliminary training step starting from training data of the training dataset, and variant_level_contribution corresponds to the first weight parameter (vlc) or to a discretization of the first weight parameter into a limited number of discrete levels;
calculating a second contribution β₂*gene_level_contribution, where β₂ is a parameter estimated in a preliminary training step starting from training data of the training dataset, and gene_level_contribution corresponds to the second weight parameter (glc) or to a discretization of the second weight parameter into a limited number of discrete levels;
calculating a third contribution β₃*phenotype_level_contribution, where β₃ is a parameter estimated in a preliminary training step starting from training data of the training dataset, and phenotype_level_contribution corresponds to the third weight parameter (phlc) or to a discretization of the third weight parameter into a limited number of discrete levels.

In this embodiment, the step of determining the digenic mechanism of the digenic combination comprises calculating the probability that the digenic mechanism is “Dual Diagnosis” by means of logistic regression, based on the aforesaid first contribution, second contribution and third contribution, and determining that the digenic mechanism is “Dual Diagnosis” if the aforesaid probability that the digenic mechanism is “Dual Diagnosis” is above a certain threshold.

According to an implementation option of such an embodiment, the step of determining the digenic mechanism of the digenic combination comprises calculating the probability that the digenic mechanism is “Dual Diagnosis” by means of logistic regression, through the formula:

$$p ( DM \vert X ) = \frac{{e}^{α + {β}_{1}^{⋆ variantLevelContribution} + {β}_{2}^{⋆ geneLevelContribution} + {β}_{3}^{⋆ phenotypeLevelContribution}}}{1 + {e}^{α + {β}_{1}^{⋆ variantLevelContribution} + {β}_{2}^{⋆ geneLevelContribution} + {β}_{3}^{⋆ phenotypeLevelContribution}}} \text{?}$$

$$\text{?} \text{indicates text missing or illegible when filed}$$

where α is a parameter estimated from the training data of the training dataset.

According to an implementation example, the aforesaid threshold is 0.5.

In accordance with an implementation option, each of the variant_level_contribution, gene_level_contribution, phenotype_level_contribution parameters corresponds to the first weight parameter, the second weight parameter and the third weight parameter, respectively.

According to another implementation option, each of the variant_level_contribution, gene_level_contribution, phenotype_level_contribution parameters corresponds to a respective discretization of the first weight parameter, the second weight parameter, the third weight parameter.

In accordance with another implementation option, each of the variant_level_contribution, gene_level_contribution, phenotype_level_contribution parameters corresponds to a respective discretization of the first weight parameter, the second weight parameter, the third weight parameter into four discrete levels: low, medium-low, medium-high, high.

As for the “logistic regression” approach, used in the embodiment of the method described above, it can be observed that logistic regression is versatile, meaning that it works with both continuous and discrete data, and it is therefore not necessary to carry out a discretization step before training the algorithm, nor before using the trained model to predict a new example.

According to further embodiments of the method, for calculating the probability that the digenic mechanism is “Dual Diagnosis”, instead of the aforementioned “Naive Bayes” or “logistic regression” approaches, other algorithms are used, such as “decision trees” or “Random Forest”-type classifiers.

The decision tree is a predictive algorithm which learns a tree structure from data which can be used for classification. As is known, in the decision tree, each “node” represents a feature, and two or more branches extend from each node depending on the different values of that feature.

The choice of the order of the “features” along the tree structure depends on some indices calculated based on training data, such as the Gini index or the entropy, which calculate how well a given feature is capable of dividing the training set into groups which are as homogeneous as possible from the point of view of the class.

Decision trees work on discretized values, meaning that at each node one of the possible paths is taken if the feature in hand has a certain value, or (if the feature has continuous values) if the value of the feature is less than or equal to X, where the threshold value X is automatically calculated by the algorithm of the decision tree from the training data. The decision tree thus automatically carries out a discretization and, unlike Naive Bayes, does not require discretizing the values of the features before training and using the classifier.

As with Naive Bayes and logistic regression, the decision tree outputs a predicted probability of classification for a given example. This is calculated by the tree as the number of training examples belonging to a certain class C present in the “leaf” node, i.e., the last node reached by the classification path for the given example, divided by the total number of training examples in the “leaf” node. In the binary case, typically when such a frequency is >=50%, then the aforesaid class C is predicted.

Starting from a set of decision trees, according to an embodiment, a “Random Forest” classifier is also used. “Random Forests” are literally “tree forests” in which the algorithm trains a number N of decision trees on a randomly selected subset of features. The final classification prediction is the majority class, predicted by most of the individual trees forming the forest, and the classification probability for class C in this case is the number of trees which “voted” for class C divided by the total number of trees N.

In accordance with an embodiment of the method, the step of determining the digenic mechanism of the digenic combination comprises classifying the digenic combination into one or more of the following classes (the meaning of which is known per se, in the technical field considered-see also the above reported description of the known art):
Dual Diagnosis (“Dual Molecular Diagnosis”);
True Digenics;
Composite or Modifier.

Further details will be provided below, by way of non-limiting example, of an embodiment of the method, based on the results of a pathogenicity prediction performed by means of a proprietary method/model, developed by the Applicant, which will be identified briefly below as “DIVAs”, and which is shown in detail in international patent application WO 2022/195507 A1 (in the name of the same Applicant).

As noted above, one of the objectives of the present invention is to develop an approach to elucidate the digenic mechanism of a predicted positive digenic pair from a digenic model (e.g., the aforesaid proprietary “DIVAs” model; however, note that the method described here is also applicable to other possible predictive models or methods which provide equivalent and/or comparable results).

Note that the method according to the present invention does not use “Explainable Artificial Intelligence”, XAI, simply to identify features relevant for classification (e.g., gene interaction, impact of variants, etc.), but also to understand the digenic mechanism of a pathogenic pair (combination), evaluating the contribution (i.e., weight) of each of the features which have been used for the evaluation of pathogenicity.

Since different digenic mechanisms (as mentioned in the previous prior art section of the description) have different features, it can be reliably assumed that the classification process of a Machine Learning algorithm for a pathogenetic instance (in this case, variant pair) is different depending on the digenic mechanism.

The “DIVAs” model takes into account 3 different groups of features to predict pathogenicity:
1. variant-level features, i.e., the pathogenicity of the variants in a pair, as defined by a score proportional to the pathogenicity defined by international guidelines for monogenic interpretation;
2. gene-level features, i.e., features which capture the gene interaction, such as the degree of co-expression of the two genes or the presence in the same biological pathways;
3. phenotype-level features which capture the phenotypic similarity between the phenotypes expressed by the patient and those which would be expected from the mutations in hand.

Therefore, the method is based on the following reasonable assumption: for a digenic combination predicted as pathogenic by “DIVAs”, the classification process is different depending on whether this pair is a “True Digenic/Composite” or a “Dual Diagnosis”.

In the first case, for example, the “DIVAs” model will have evaluated as important for the classification the “features” which indicate gene interaction, while in the second case it will have carried out the classification based on the high pathogenicity of the variants forming the pair.

In the specific example shown here, the following approach was taken.

For each pair classified as pathogenic by the “DIVAs” model, the classification process is evaluated by means of Explainable Artificial Intelligence, XAI.

In particular, in this example, SHAP is used, i.e., a widely used local XAI method, which assigns each feature used by “DIVAs” a numerical weight (the aforementioned “Shapley value”) which represents the contribution of that feature to the probability of pathogenicity predicted by “DIVAs”. The higher the Shapley value for a given feature, the more important the value of that feature for the final classification.

A characteristic aspect of the Shapley values is that the sum thereof is equivalent to the predicted probability.

Once the Shapley value has been calculated for each feature, the Shapley values of the features belonging to the same category (variant-level contribution, or gene-level contribution, or phenotype-level contribution) are added

On a training dataset, it has been demonstrated that the importance of the features for the classification (measured in terms of Shapley values) is different depending on the digenic mechanism (for example, for Dual Diagnoses, the weight of variant-level features is much greater than the weight calculated in True Digenics/Composites).

Once the contributions of the features for the classification in the form of Shapley value have been calculated, such values are discretized into 4 levels (e.g., low, low-median, median-high, high) for each sub-group of features (variant-level contribution, gene-level contribution, and phenotype-level contribution) based on thresholds calculated on the training dataset (for example, calculating the first, second and third quartiles).

For example, a predicted pathogenic combination can have variant-level contribution=low, gene-level contribution=high and phenotype-level contribution=median-high.

The probabilities of being a Dual Diagnosis are then calculated on the training dataset given the discrete values of variant-level contribution, phenotype-level contribution and gene-level contribution, by means of a known Machine Learning approach called Naive Bayes (already mentioned above—see also:
https://en.wikipedia.org/wiki/Naive_Bayes_classifier
   https://www.saedsayad.com/naive_bayesian.htm),
and in particular the formula [1] already previously reported in this description, where p(DM) is the probability that a pair is a Dual Diagnosis, also calculated on a training dataset.

Therefore, each time a new pair is classified as pathogenic by the “DIVAs” model, the contributions of each feature to the classification by means of SHAP are calculated and discretized, and then the probability that the pair is Dual Diagnosis is calculated according to the equation above.

If such a probability is >=50% then the pair is considered Dual Diagnosis, otherwise it is considered True Digenic/Composite.

It can be noted, therefore, that the present method does not develop an ad-hoc classifier to distinguish the digenic mechanism, but uses the classification pattern of a pathogenicity prediction model (in this example, the “DIVAs” model).

Moreover, the present method uses “Explainable Artificial Intelligence” (XAI) not to select important features before developing the classifier, or to show the user the importance of the features in a classification, but instead employs “Explainable Artificial Intelligence” as features to further predict the sub-classification of digenic variants depending on the digenic mechanism underlying them.

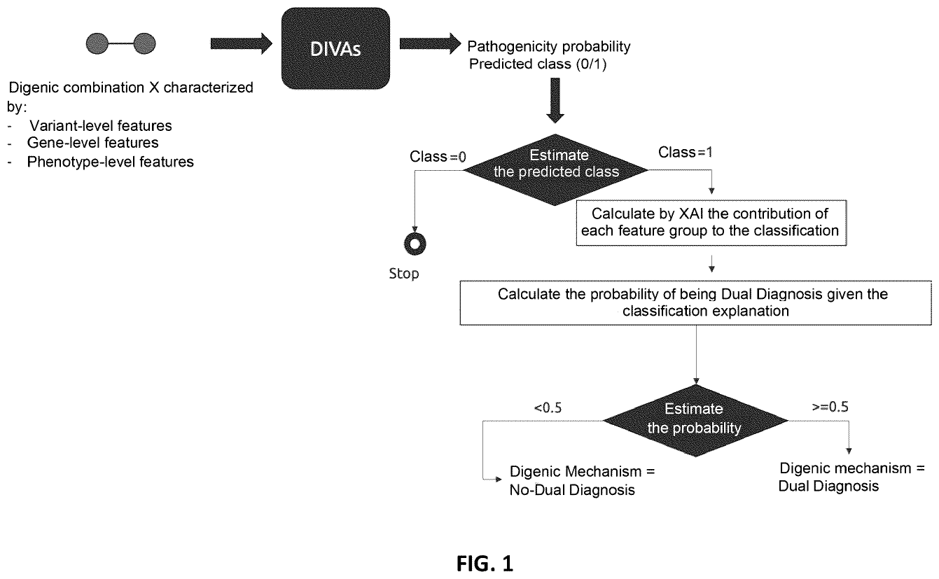

According to a particular implementation, shown in FIG. 1, given a pair of variants in a digenic combination, characterized by a set of features, the “DIVAs” predictive model of pathogenicity predicts the probability that the instance is pathogenic based on such features.

If such a probability is high, the instance is assigned class 1 (pathogenic), otherwise class 0 (benign) is assigned.

If the predicted class is 1 (pathogenic), the contributions of each feature to the classification are calculated by means of XAI (SHAP) and these contributions are used, as mentioned above, as features for a Naive Bayes classifier, to assign the probability of being “Dual Diagnosis” based on the DIVAs classification process.

The assumption behind the Naive Bayes approach is that the probability of a class is proportional to the production of conditional probabilities on each individual feature.

In detail, given a training dataset Xₜ=x₁ . . . xₚ . . . xₙ . . . xₚ where each example xᵢ is characterized by a set of p categorical features and belongs to a given class, the probability that a hypothetical new example \hat{x} belongs to class C is calculated according to the following formula:

$$p ( C \vert \hat{x} ) ∼ p ( C \vert feature\_ 1 = {\hat{x}}_{1} ) * p ( C \vert feature\_ 2 = {\hat{x}}_{2} ) * …$$

The probability of class C given a particular value of a feature (i.e., every single element of the production) is calculated as the frequency of training examples belonging to class C and having exactly that value of the feature.

In the example shown here, a binary classification (DM/No-DM) must be performed based on the values of three features: gene_level_contribution, variant_level_contribution, phenotype_level_contribution.

Since these features are numerical, as they represent Shapley's values, while on the other hand the Naive Bayes works with categorical and non-numerical features, the possible values of each feature are discretized into low, low-median, median-high, and high, based on the comparison of the value of that feature with the percentiles calculated on the training dataset.

Suppose having this type of training dataset available:

| | gene_level | Variant_level | pheno_level | class |
|---|---|---|---|---|
| V1 | Low | High | Low | DM |
| V2 | Low-median | Low | High | No-DM |
| V3 | Low-median | Median-high | Low | DM |
| V4 | High | Median-high | Median-high | No-DM |
| V5 | High | Median-high | Median-high | No-DM |
| V6 | Low | High | Low-median | DM |
| V7 | Low-median | Low-median | Low | No-DM |

The first example in the training belongs to the class DM, and has low gene level, high variant level and low phenotype level.

The Naive Bayes formula in this case is as follows:

$$p ( DM \vert {X}_{i} ) ∼ p ( DM ) * \frac{p ( DM \vert gene\_level {\_contribution}_{i} )}{p ( DM )} * \frac{p ( DM \vert variant\_level {\_contribution}_{i} )}{p ( DM )} * \frac{p ( DM \vert pheno\_level {\_contribution}_{i} )}{p ( DM )}$$

The method includes learning every single probability reported in the aforesaid formula 5 from the training data.

For example, P(DM)=0.43, as we have 3 out of 7 cases in the training which belong to the class DM.

As for conditional probabilities, p(C=DM|gene_level_contribution-low)=2/3=0.67, i.e., the frequency of examples DM among those with gene_level_contribution equal to low.

By repeating the calculation for each feature and for each possible value of that feature, the following table of conditional probabilities is obtained.

| | | | P(No-DM\|Attribute = |
|---|---|---|---|
| Attribute | Value | P(DM\|Attribute = value) | value) |
| gene_level_{—} | low | 2/2 = 1 | 0 |
| contribution | low-median | ⅓ = 0.33 | ⅔ = 0.67 |
| | median-high | 0 | 0 |
| | high | 0 | 1 |
| variant_level_{—} | low | 0 | 1 |
| contribution | low-median | 0 | 1 |
| | median-high | ⅓ = 0.33 | 0.67 |
| | high | 1 | 0 |
| phenotype_level_{—} | low | ⅔ = 0.67 | ⅓ = 0.33 |
| contribution | low-median | 1 | 0 |
| | median-high | 0 | 1 |
| | high | 0 | 1 |

Now suppose having to predict a new example, which has gene_level=low-median, variant_level=median_high and phenotype_level=low.

In this case, the predicted probability of the class DM will be proportional to:

$$P ( X ) ∼ 0.43 * \frac{0.33}{0.43} * \frac{0.33}{0.43} * \frac{0.67}{0.43} = 0.39$$

while the probability of No-DM will be proportional to:

$$P ( X ) ∼ 0.57 ⋆ \frac{0.67}{0.57} ⋆ \frac{0.67}{0.57} ⋆ \frac{0.33}{0.57} = 0.45$$

The highest estimate is that of the class No-DM. To calculate a probability value between 0 and 1, it can be normalized as follows:

$$p ( X ) = \frac{0.39}{0.39 + 0.45} = 0.46$$

A computer-implemented method for determining the pathogenicity of combinations of digenic variants and classifying digenic variants recognized as pathogenic is described here, which is also comprised in the present invention. Such a method comprises the following steps:
defining a set of variants, the pathogenicity of which must be determined, in which such variants refer to mutations present in one or both alleles of a respective gene of at least two genes, each of the genes being associated with two respective alleles;
determining situations which can occur, regarding the presence or absence of said variants in the alleles of said at least two genes, wherein each situation is associated with a respective combination in which each variant is present in a respective subset of alleles, among all the possible subsets of alleles of all the genes considered, or it is present in all the alleles of all the genes considered;
for each of said defined situations, i.e., for each combination and each gene, calculating a pathogenicity index or score, adapted to estimate how much the respective variants singularly modify the functioning of the respective gene;
describing phenotypic traits of a patient, by standardized phenotypic terms, i.e., standardized information adapted to describe phenotypic anomalies found in the patient;
calculating or preparing input information for the pathogenicity determination of each combination, comprising:
variant-level features, representative of the pathogenicity of the single variants in the digenic combination;
gene-level features, representative of the interaction between the genes forming the digenic combination and/or a priori property features of each of the two genes of the digenic combination;
phenotype-level features, representative of gene-phenotype association, calculated individually for each of the genes considered, and adapted to measure how much phenotypic traits of the patient are superimposable to phenotypes already known to be associated with the single gene;
providing the aforesaid input information for the pathogenicity determination to at least one trained algorithm;
processing the aforesaid input information for the pathogenicity determination by the at least trained algorithm.

The aforesaid trained algorithm is an algorithm trained by artificial intelligence and/or machine learning techniques.

Such an algorithm is trained in a preliminary training step, based on a training dataset of known cases, providing the aforesaid input information calculated for each of the known cases to the algorithm to be trained, and training the algorithm based on the knowledge of the pathogenicity/benignity of the respective known digenic cases.

The method then comprises the further steps of obtaining output information from the trained algorithm, representing the pathogenicity of the combination of digenic variants or mutations considered, and performing a method for determining the digenic mechanism of a digenic combination recognized as pathogenic according to any of the embodiments of such a method described above.

According to an embodiment, the methods according to the invention, previously mentioned, are carried out by means of one or more computers, in which programs, implementing the algorithms and models mentioned above, are loaded and executable.

As it can be seen, the objects of the present invention as previously indicated are fully achieved by the method described above by virtue of the features shown above in detail.

In order to meet contingent needs, those skilled in the art may make changes and adaptations to the embodiments of the method described above or can replace elements with others which are functionally equivalent, without departing from the scope of the following claims. Each of the features described above as belonging to a possible embodiment can be implemented irrespective of the other embodiments described.

1. A computer-implemented method for determining a digenic mechanism of a digenic combination recognized as pathogenic in relation to a set of phenotypes observed in an individual, wherein the digenic combination refers to variants related to two genes, referring to mutations present in one or both alleles of a respective gene of at least two genes, each of the genes being associated with two respective alleles,
wherein the digenic combination is recognized as pathogenic based on a pathogenicity determination carried out by a trained algorithm trained by artificial intelligence and/or machine learning techniques based on a training dataset of known cases, wherein the pathogenicity determination is based on at least the following features:
variant-level features, representative of the pathogenicity of the single variants forming the digenic combination;
gene-level features, representative of interaction between the genes forming the digenic combination and/or a priori property features of each of the two genes of the digenic pair;
phenotype-level features, representative of gene-phenotype association, calculated individually for each of the genes considered, and adapted to measure how much the phenotypic traits of the patient are superimposable to phenotypes already known to be associated with the single gene;
wherein the recognition of the digenic mechanism comprises recognizing whether the digenic mechanism is “Dual Diagnosis” or not, the digenic mechanism being “Dual Diagnosis” if each mutated gene individually causes a respective disease;
wherein the method comprises:
determining, by Explainable Artificial Intelligence, XAI, techniques, a first weight parameter representative of the contribution of the variant-level features to the pathogenicity determination of the digenic combination, a second weight parameter representative of the contribution of the gene-level features to the pathogenicity determination of the digenic combination, a third weight parameter representative of the contribution of the phenotype-level features to the pathogenicity determination of the digenic combination;
determining, by machine learning techniques or models, the digenic mechanism of the digenic combination, and then recognizing whether the digenic mechanism is “Dual Diagnosis” or not, based on said first weight parameter, second weight parameter and third weight parameter determined by Explainable Artificial Intelligence, XAI, techniques.

2. A method according to claim 1, wherein said step of determining, by Explainable Artificial Intelligence, XAI, techniques, a first weight parameter, a second weight parameter and a third weight parameter comprises:
calculating the first weight parameter as the Shapley value of the variant-level features, indicative of a contribution of the variant-level features to the predicted pathogenicity probability of the digenic combination;
calculating the second weight parameter as the Shapley value of the gene-level features, indicative of a contribution of the gene-level features to predicted pathogenicity probability of the digenic combination;
calculating the third weight parameter as the Shapley value of the phenotype-level features, indicative of a contribution of the phenotype-level features to the predicted pathogenicity probability of the digenic combination.

3. A method according to claim 1, further comprising the steps of:
calculating, in a preliminary training step on a training dataset, a first conditional probability of “Dual Diagnosis” digenic combination p(DM|variant_level_contribution) based on the frequency of digenic training combinations indicated in the training dataset as of “Dual Diagnosis” type given a first parameter value variant_level_contribution corresponding to the first weight parameter, for each digenic training combination belonging to the training dataset, or a discretization of said first weight parameter into a limited number of discrete levels;
calculating, in a preliminary training step on the training dataset, a second conditional probability of “Dual Diagnosis” digenic combination p(DM|gene_level_contribution) based on the frequency of digenic training combinations indicated in the training dataset as of “Dual Diagnosis” type given a second parameter value gene_level_contribution corresponding to the second weight parameter, for each digenic training combination belonging to the training dataset, or a discretization of said second weight parameter into a limited number of discrete levels;
calculating, in a preliminary training step on the training dataset, a third conditional probability of “Dual Diagnosis” digenic combination p(DM|phenotype_level_contribution) based on the frequency of digenic training combinations indicated in the training dataset as of “Dual Diagnosis” type given a third parameter value phenotype_level_contribution corresponding to the third weight parameter, for each digenic training combination belonging to the training dataset, or a discretization of said third parameter into a limited number of discrete levels; and
wherein the step of determining the digenic mechanism of a digenic combination to be classified, in an operating step of the method, comprises determining the digenic mechanism of the digenic combination based on said first conditional probability, second conditional probability, and third conditional probability.

4. A method according to claim 3, wherein the step of determining the digenic mechanism of the digenic combination comprises:
calculating a score proportional to the probability that the digenic mechanism is “Dual Diagnosis” by a Naive Bayes-type machine-learning model, based on the training dataset, through the formula:
$$p ( DM \vert {X}_{i} ) \sim p ( DM ) * \frac{p ( DM \vert gene\_level {\_contribution}_{i} )}{p ( DM )} * \frac{p ( DM \vert variant\_level {\_contribution}_{i} )}{p ( DM )} * \frac{p ( DM \vert pheno\_level {\_contribution}_{i} )}{p ( DM )}$$
where p(DM) is the total probability that the digenic mechanism is “Dual Diagnosis” calculated on the training dataset as well,
where variant_level_contribution corresponds to the first weight parameter or a discretization of the first weight parameter into a limited number of discrete levels, gene_level_contribution corresponds to the second weight parameter or a discretization of the second weight parameter into a limited number of discrete levels, phenotype_level_contribution corresponds to the third weight parameter or a discretization of the third weight parameter into a limited number of discrete levels;
determining that the digenic mechanism is “Dual Diagnosis” if said score proportional to the probability P (DM|Xi) that the digenic mechanism is “Dual Diagnosis” is above a certain threshold.

5. A method according to claim 4, wherein the predicted score is normalized between 0 and 1, to obtain a normalized score (P_{FINAL}), by the following formula:
$${P}_{FINAL} ( DM \vert {X}_{i} ) = \frac{P > ( DM \vert {X}_{i} )}{P ( DM \vert {X}_{i} ) + P ( NoDM \vert {X}_{i} )}$$
and wherein said step of determining that the digenic mechanism is “Dual Diagnosis” comprises comparing said normalized score (P_{FINAL}) with a normalized threshold.

6. A method according to claim 5, wherein said normalized threshold is 0.5.

7. A method according to claim 4, wherein each of variant_level_contribution, gene_level_contribution, phenotype_level_contribution corresponds to a respective discretization of the first weight parameter, the second weight parameter, or the third weight parameter.

8. A method according to claim 7, wherein each of variant_level_contribution, gene_level_contribution, phenotype_level_contribution corresponds to a respective discretization of the first weight parameter, the second weight parameter, or the third weight parameter into four discrete levels: low, medium-low, medium-high, high.

9. A method according to claim 1, further comprising the steps of:
calculating a first contribution β₁*variant_level_contribution, where β₁ is a parameter estimated in a preliminary training step starting from training data of the training dataset, and variant_level_contribution corresponds to the first weight parameter or to a discretization of the first weight parameter into a limited number of discrete levels;
calculating a second contribution β₂*gene_level_contribution, where β₂ is a parameter estimated in a preliminary training step starting from training data of the training dataset, and gene_level_contribution corresponds to the second weight parameter or to a discretization of the second weight parameter into a limited number of discrete levels;
calculating a third contribution β₃*phenotype_level_contribution, where β₃ is a parameter estimated in a preliminary training step starting from training data of the training dataset, and phenotype_level_contribution corresponds to the third weight parameter or to a discretization of the third weight parameter into a limited number of discrete levels; and
wherein the step of determining the digenic mechanism of the digenic combination comprises:
calculating the probability that the digenic mechanism is “Dual Diagnosis” by logistic regression, based on said first contribution, second contribution and third contribution;
determining that the digenic mechanism is “Dual Diagnosis” if said probability that the digenic mechanism is “Dual Diagnosis” is above a certain threshold.

10. A method according to claim 9, wherein the step of determining the digenic mechanism of the digenic combination comprises:
calculating the probability that the digenic mechanism is “Dual Diagnosis” by logistic regression, through the formula:
$$p ( DM \vert X ) = \frac{{e}^{α + {β}_{1}^{⋆ variantLevelContribution} + {β}_{2}^{⋆ geneLevelContribution} + {β}_{3}^{⋆ phenotypeLevelContribution}}}{1 + {e}^{α + {β}_{1}^{⋆ variantLevelContribution} + {β}_{2}^{⋆ geneLevelContribution} + {β}_{3}^{⋆ phenotypeLevelContribution}}} \text{?}$$
$$\text{?} \text{indicates text missing or illegible when filed}$$
where α is a parameter estimated starting from the training data of the training dataset.

11. A method according to claim 10, wherein said threshold is 0.5.

12. A method according to claim 10, wherein each of variant_level_contribution, gene_level_contribution, phenotype_level_contribution corresponds to the first weight parameter, the second weight parameter and the third weight parameter, respectively.

13. A method according to claim 10, wherein each of variant_level_contribution, gene_level_contribution, phenotype_level_contribution corresponds to a respective discretization of the first weight parameter, the second weight parameter, the third weight parameter.

14. A method according to claim 13, wherein each of variant_level_contribution, gene_level_contribution, phenotype_level_contribution corresponds to a respective discretization of the first weight parameter, the second weight parameter, the third weight parameter into four discrete levels: low, medium-low, medium-high, high.

15. A method according to claim 1, wherein the step of determining the digenic mechanism of the digenic combination comprises classifying the digenic combination into one or more of the following classes:
Dual Molecular Diagnosis;
True Digenics;
Composite or Modifier.

16. A computer-implemented method for determining pathogenicity of combinations of digenic variants and classifying digenic variants recognized as pathogenic, comprising the steps of:
defining a set of variants, the pathogenicity of which must be determined, wherein said variants refer to mutations present in one or both alleles of a respective gene of at least two genes, each of the genes being associated with two respective alleles;
determining situations which can occur, regarding the presence or absence of said variants in the alleles of said at least two genes, wherein each situation is associated with a respective combination in which each variant is present in a respective subset of alleles, among all possible subsets of alleles of all the genes considered, or each variant is present in all the alleles of all the genes considered;
for each of said defined situations, comprising each combination and each gene, calculating a pathogenicity index or score, adapted to estimate how much the respective variants singularly modify the functioning of the respective gene;
describing phenotypic traits of a patient, by standardized phenotypic terms, comprising standardized information adapted to describe phenotypic anomalies found in the patient;
calculating or preparing input information for the pathogenicity determination of each combination, comprising:
variant-level features, representative of the pathogenicity of the single variants in the digenic combination;
gene-level features, representative of interaction between the genes forming the digenic combination and/or a priori property features of each of the two genes of the digenic combination;
phenotype-level features, representative of gene-phenotype association, calculated individually for each of the genes considered, and adapted to measure how much phenotypic traits of the patient are superimposable to phenotypes already known to be associated with the single gene;
providing said input information for the pathogenicity determination to at least one trained algorithm;
processing said input information for the pathogenicity determination by the at least trained algorithm,
wherein said trained algorithm is an algorithm trained by artificial intelligence and/or machine learning techniques,
wherein said algorithm is trained in a preliminary training step, based on a training dataset of known cases, providing said input information calculated for each of the known cases to the algorithm to be trained, and training the algorithm based on the knowledge of the pathogenicity/benignity of the respective known digenic cases;
obtaining output information from the trained algorithm, representing the pathogenicity of the combination of digenic variants or mutations considered;
performing a method for determining the digenic mechanism of a digenic combination recognized as pathogenic according to claim 1.
